(19) United States
(12) Patent Application Publication
(10) Pub. No.: US 2010/0228135 A1
(43) Pub. Date: Sep. 9, 2010

(54) IMPLANTABLE PERFUSION SENSOR

(75) Inventors: Randal C. Schulhauser, Phoenix, AZ (US); Matthew C. Bond, Phoenix, AZ (US); Michael R. Kane, Pewaukee, WI (US)

Correspondence Address: MEDTRONIC, INC., 710 MEDTRONIC PARKWAY NE, MINNEAPOLIS, MN, 55432-9924, US

(73) Assignee: Medtronic, Inc.

(21) Appl. No.: 12/757,158

(22) Filed: Apr. 9, 2010

Related U.S. Application Data
(62) Division of application No. 11/323,015, filed on Dec. 30, 2005, now abandoned.

(51) Int. Cl. A61B 5/026

(52) U.S. Cl. 600/505

(57) ABSTRACT

An injectable wireless perfusion sensor provides data to an external device regarding the perfusion of the targeted tissue. The sensor permits a caregiver to monitor cardiovascular performance in specific areas such as the extremities. The sensor will identify whether vascular constriction or obstruction is present and to what extent. Further, once such a condition is treated, the sensor will monitor the effectiveness of that treatment.

### RELATED APPLICATION

This application is a division of and claims priority to U.S. patent application Ser. No. 11/323,015, Schulhauser et al, Implantable Perfusion Sensor, filed Dec. 30, 2005.

### BACKGROUND

1. Field of the Invention

The present invention relates to implantable medical devices and more specifically to implantable medical devices having sensing capabilities.

2. Description of the Related Art

Peripheral Vascular Disease (PVD) refers to a number of conditions that occur within the vasculature generally outside of the heart and the brain. There are two broad categories of PVD including transient variants such as Raynaud's disease and structural variants resulting from occlusion, inflammation or tissue damage affecting a vascular structure. Peripheral artery disease (PAD) is an example of such a variant and is caused by partial or complete occlusion. PAD is similar to coronary artery disease (CAD), which occurs in and around the vasculature of the heart. When the occlusion becomes sufficient, ischemia results leading to an infarct if alternative delivery pathways are not available.

Transient varieties of PVD, such as Raynaud's disease relate to spasmic occlusion of a vessel, often induced by exposing an extremity to a cold source. The patient's reaction to the low temperature causes vasoconstriction that reduces or prevents blood flow. Stress and other factors may induce episodes. Typically, the episodes are transient and upon vasodilation blood flow resumes without damage to the relevant tissue. In some cases, an infarct may occur that results in the loss of tissue or a digit of the extremities. The patient is particularly sensitive to cold and the phenomenon of vasoconstriction and subsequent is particularly sensitive to cold and the phenomenon of vasoconstriction and subsequent dilation results in particular tonal changes to the skin that evidence Raynaud's disease. Treatment generally includes avoiding exposure to the cold and in severe cases drugs may be provided.

With PAD and similar variants, there is a wide variety of symptoms with many patients being completely unaware that any condition exists. Diabetic patients are at increased risk for the disease as well as for developing serious complications from the disease. As indicated, as many as half the patient population with PAD may be asymptomatic. Those patients experiencing symptoms may feel fatigue, pain, cramping, numbness, or a feeling of coldness. Arterial occlusions may lead to ischemia and infarct that result in the loss of a limb and may have fatal consequences.

Of note, PAD is similar to CAD. Furthermore, a finding of PAD may indicate CAD and cardiac disorders that cause emboli, such as atrial fibrillation, are a major cause of arterial embolisms resulting in PAD. Thus, patients having CAD, various cardiac disorders, and diabetes are at particular risk for PAD and may receive heightened scrutiny during a medical exam. Conversely, a determination of PAD may lead to testing for previously unknown CAD, cardiac disorders and diabetes.

When suspected, the patient's pulse is evaluated distal to the occlusion. If present, the pulse is reduced or absent. Pressure measurements are evaluated, such as with the ankle brachial index. Sequentially obtained (distally) measurements may be made to identify the location and the severity of the obstruction, when present in an extremity. MRI, Doppler measurement and other non-invasive techniques may be employed for further diagnosis.

Treatment will depend upon the severity of the disease and range from monitoring the status to providing medications, such as blood thinners, to more invasive techniques such as implanting a stent, atherectomy, bypass procedures and if necessary, amputation.

### BRIEF DESCRIPTION OF THE DRAWINGS

FIGS. 1A and 1B illustrate the selected anatomical features.

FIGS. 2A and 2B illustrate the placement of wireless perfusion sensors in selected anatomical locations.

FIGS. 3A and 3B illustrate a perfusion sensor.

FIG. 4 is a schematic diagram illustrating selected components of a wireless perfusion sensor.

FIGS. 5 and 6 illustrate a wireless perfusion sensor.

FIG. 7 schematically illustrates the data exchange between a wireless perfusion sensor and various external devices.

### DETAILED DESCRIPTION

The entire content of U.S. application Ser. No. 11/323,015, filed Dec. 30, 2005, is hereby incorporated by reference.

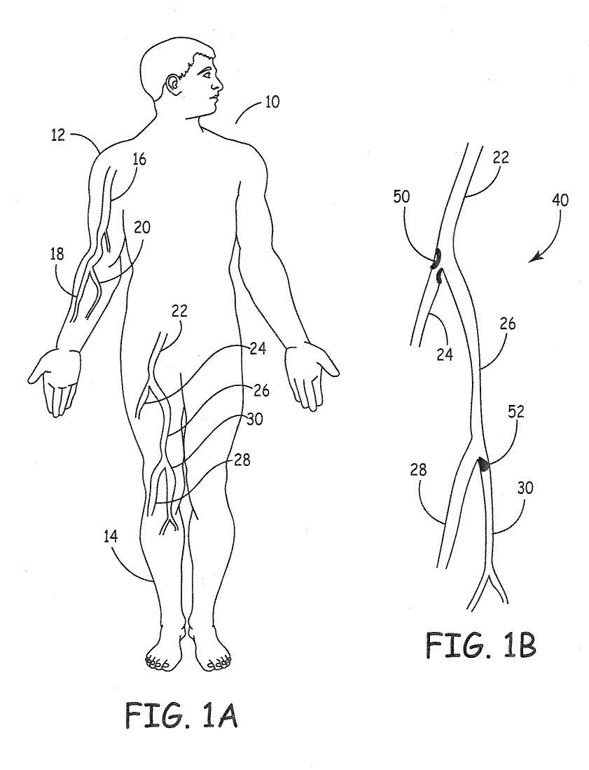

FIGS. 1A and 1B illustrate a patient 10 and certain portions of the vasculature anatomy. It should be appreciated that such anatomical representations are for illustrative purposes only and not necessarily accurate. In the right arm 12 of the patient 10, the brachial artery 16 is illustrated, along with the radial artery 18 and the ulnar artery 20. In the right leg 14 of the patient 10, the following arteries are illustrated: the external iliac 22, deep femoral 24, femoral 26, popliteal 28 and the genicular artery 30. FIG. 1B is an enlargement of the lower extremity limb vasculature 40 illustrated in the right leg 14 in FIG. 1A. A partial occlusion 50 is illustrated at the proximal end of the deep femoral artery 24. Thus, blood flow through the deep femoral artery 24 and any dependent arteries will be reduced. A complete occlusion 52 is illustrated at the genicular artery; thus, blood flow is completely stopped beyond this point.

The occlusions 50, 52 may be formed in any number of ways. For example, a thrombus may be present, which is essentially a blood clot. Plaque or cholesterol deposits may also form occlusions. There may be a stricture, which is a scarring of the artery that forms as the result of some insult or injury. Alternatively, the occlusion 50, 52 is the result of external pressure or constriction, rather than a foreign body. Of course, there are a great many things that may result in the occlusions 50, 52 with the net result being either a reduction (which may or may not be significant) or cessation of blood flow beyond the occlusion.

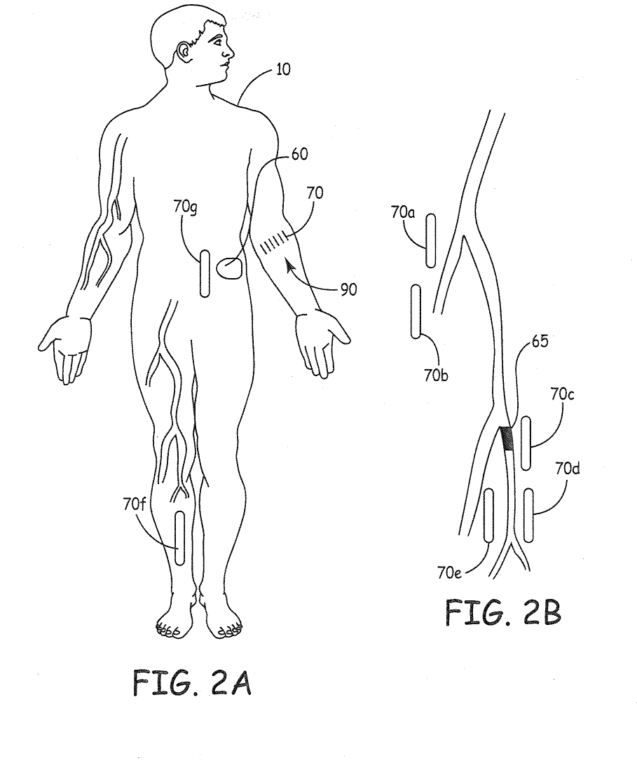

Depending upon the nature and severity of the occlusion 50, 52, the patient 10 may or may not experience symptoms and if symptoms are present, they may be transient or of such a nature (e.g., numbness, tingling) that they are ignored. Alternatively, the patient's caregiver may identify the occlusions 50, 52 and provide an appropriate course of treatment. FIGS. 2A and 2B illustrate the patient 10 post treatment. As illustrated, both occlusions 50, 52 have been eliminated. Treatments may include medications to break up the occlusion or blood thinners to facilitate passage through constricted vasculature (in which case, the occlusion may still be present, but its effects are reduced). An atherectomy may be performed to mechanically remove the occlusion (e.g., angioplasty, laser removal, drilling/cutting, or traditional surgical removal). Another alternative is to perform a bypass procedure wherein vasculature from a donor or another portion of the patient 10 is used to circumvent the occlusion. With any of these procedures or as an independent treatment, a stent 65 may be implanted at the site to support the vasculature. Such a stent 65 may be uncoated or may be drug eluting.

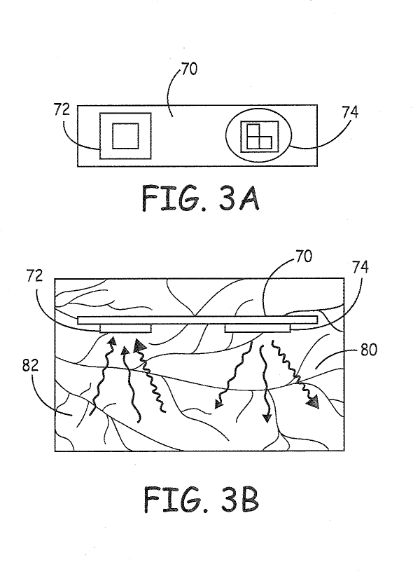

In general, PVD is not well understood and despite the use of these treatments, recurrence is often likely. It is also difficult to gauge any degree of success or predict which patients are likely to develop subsequent, similar problems. FIGS. 3A and 3B illustrate a perfusion sensor 70. The perfusions sensor 70 has one or more light-emitting diodes (LED) 74 (or other light sources) and one or more photodetectors 72. FIG. 3A illustrates a bottom planar view of the perfusion sensor 70 and FIG. 3B illustrates the sensor 70 positioned such that the emitter 74 and photodetector 72 are proximate or in contact with tissue 80. The tissue 80 is supplied with oxygenated blood by a plurality of capillaries 82, which are ultimately supplied by a given artery.

As tissue 80 is deprived of oxygen, the color of the tissue varies. Thus, the perfusion sensor 70 is able to determine how oxygenated the relevant tissue 80 is based upon any color or tonal changes. Of course, human physiology delivers oxygenated blood in a pulsitile manner, thus, there will be normal or expected variations. However, if the blood supply to the tissue is compromised a notable change will occur relatively rapidly. The degree to which the supply is compromised may also be determined; that is, perfusion may be minimally impacted or there may be tissue death, with a spectrum between.

Referring to FIGS. 2A and 2B, a number of perfusion sensors 70 are illustrated, though for illustrative purposes not to scale. Sensor 70a is positioned proximate the site of former partial occlusion 50. These perfusion sensors 70 are small in scale and may be injectable through traditional means (e.g., hypodermic needle, catheter delivery, etc.) into tissue. As explained above, the perfusion sensor 70 may be positioned to monitor tissue proximate a desired location; alternatively, the sensor 70 may be positioned such that the artery in question and more specifically the site of the occlusion is monitored. That is, light is directed to the selected location of the artery and the reflected light is indicative of oxygen saturation. With the monitoring of a specific occlusion, other techniques such as Doppler or acoustic techniques may be used to measure the dimensions of the artery.

Positioning a relatively small perfusion sensor 70a at exactly the location of the occlusion 50 and achieving proper orientation of the emitter 74 and detector 72 may be challenging. Thus, the present inventions also provides for implanting or injecting one or more perfusion sensors 70b into tissue (or monitoring the artery itself) distal to the occlusion site. Thus, if the occlusion 50 were to reform (or not have been completely removed in the first place), the effect will be apparent to such sensors 70b as the perfusion of tissue is affected. Similarly, sensor 70c is illustrated proximate the stent 65. While achieving exact placement may still be challenging, the sensor 70c may be implanted concurrent with the stent 65 thus making placement somewhat less difficult. Furthermore, the sensor 70c, in one embodiment may be incorporated into a portion of the stent 65. Sensors 70d and 70e are positioned to monitor tissue distal to the stent 65 such that if the stent fails and blood flow is occluded, the sensors 70 will determine this effect on tissue perfusion.

Sensor 70f represents another distal implant site. Thus far, the sensors 70 have been presented as a mechanism to monitor the success of a given treatment for a known occlusion. It should be appreciated that with the size and ease of implant of the perfusion sensor 70, such a sensor may be used to determine whether an occlusion is present, if so where, and to what extent the occlusion is impeding circulation. For example, sensor 70f may be used to determine the effectiveness of stent 65 or prior to such treatment, that sensor may have been implanted and identified that an occlusion was present proximal to the sensor 70f.

A single implanted sensor 70 may or may not be sufficient for either monitoring treatment or detecting circulation problems. That is, a single sensor 70 implanted at the correct location will identify variations in tissue perfusion. However, the selected tissue may be supplied by multiple arteries or an artery not affected by a particular occlusion. In such a case, an array 90 of perfusion sensors 70 is implanted or injected. The number of such sensors 70 in a given array 90 will vary, but they are positioned to cover a larger area of a limb so that any circulation abnormalities will likely affect some area of tissue under surveillance. Thus, while illustrated as a single sensor 70f, an array 90 may be placed about the leg at one or more depths.

Even when a given occlusion is identified and treated, other occlusions may be present. Thus, a caregiver may remove a known occlusion and restore circulation; however, another occlusion may have escaped detection and either pose an immediate problem or worsen over time. The sensor 70 or an array 90 may be used to monitor the success of the given treatment and also may serve to identify the presence of additional occlusions.

With one or more sensors 70 so implanted, tissue perfusion is monitored for a given patient. Thus, the success of a given treatment can be evaluated and if necessary further or alternate treatments may be provided. Without such monitoring, an occlusion may worsen and lead to the loss of tissue, the amputation of a limb or potentially even death. Conversely, when a patient 10 has symptoms that are not readily explained, such monitoring may be provided to identify transient occlusions or static occlusions that were previously unknown or undetectable.

FIG. 2A illustrates a perfusion sensor 70g (which could also be an array 90) positioned proximate a given organ 60 (e.g., liver) within the patient 10. The perfusion sensor 70g is used to determine the general status of the organ 60. For example, a given patient 10 may have liver damage 10. Detected variations in perfusion may be an early indicator if imminent organ failure and may alert the patient or caregiver so that action is taken. Likewise, after an organ transplant, the transplanted organ (e.g., liver 60) is monitored via perfusion sensor 70g. Variations in perfusion may indicate that the organ is being rejected. Treatment may include varying the patient's anti-rejection medication or determining that another transplant is necessary.

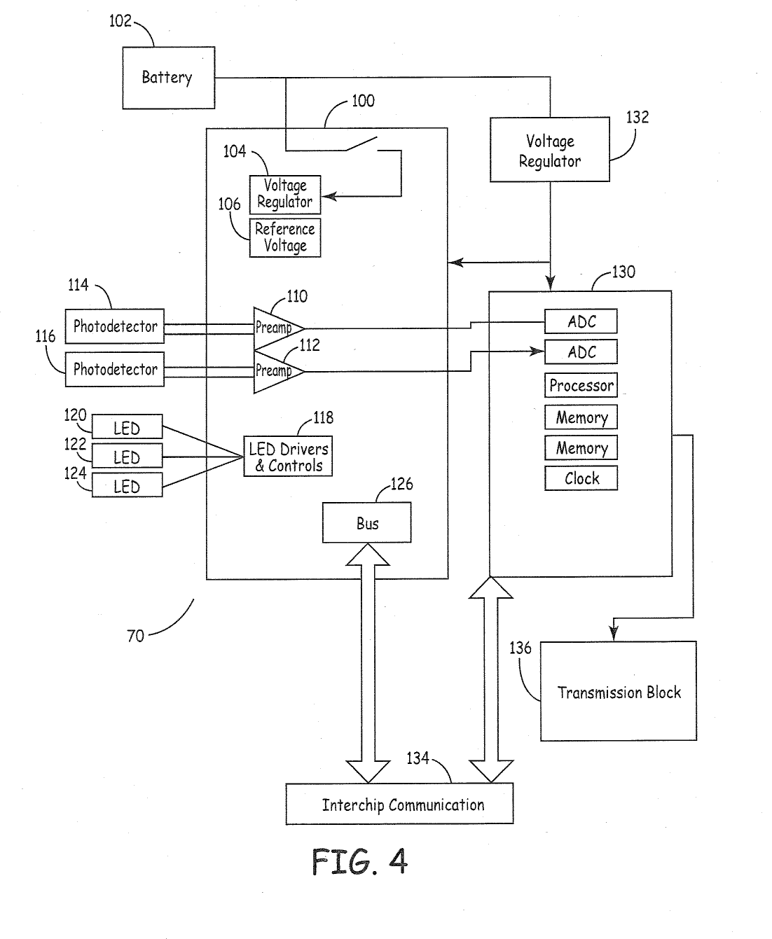

FIG. 4 is a schematic diagram of one embodiment of the perfusion sensor 70. A mixed signal chip 100 is coupled with a power source 102 such as a battery. Power is controlled through a voltage regulator 104 and a reference voltage 106 is provided. One or more photodetectors 114, 116 are provided and their output is directed to one or more amplifiers 110, 112. An LED driver 118 is provided and is coupled with one or more LEDs 120, 122, 124. It should be appreciated that light may be generated at one, two, three or more specific wavelengths and that the photodetectors 114, 116 provided will detect such wavelengths. The use of multiple wavelengths allows perfusion to be determined more accurately. It should further be appreciated, that in any given sensor 70 there may be multiple LEDs and multiple photodetectors provided at various locations to provide redundancy and reduce the need to control the orientation of the sensor 70 at implant. Furthermore, after implant the user may elect to enable or disable some of these LEDs and corresponding photodetectors. Each photodetector 114, 116 may be dedicated to a specific wavelength. Alternatively, each photodetector 114, 116 detects multiple wavelengths and this allows for greater conservation of space.

A communication bus 126 is provided to enable data transfer via interchip communication module 134. A microprocessor 130 is powered by the power source 102 via voltage regulator 132. The microprocessor 130 includes one or more analog to digital converters that receive the amplified output from the photodetectors, one or more memory devices and a clock. The signal from the photodetectors is processed and an output indicative of tissue color/perfusion or changes thereto is stored in the memory. While not limiting, in one embodiment sufficient memory is provided to collect data for about 100 days. The sensor 70 includes a transmission module 136 that permits the data within the memory to be telemetered to an external device wherein a caregiver may evaluate the data. Such transmission may occur on a real-time basis to an external device that is maintained proximate the patient 10 or on a periodic basis when the external device interrogates the sensor 70.

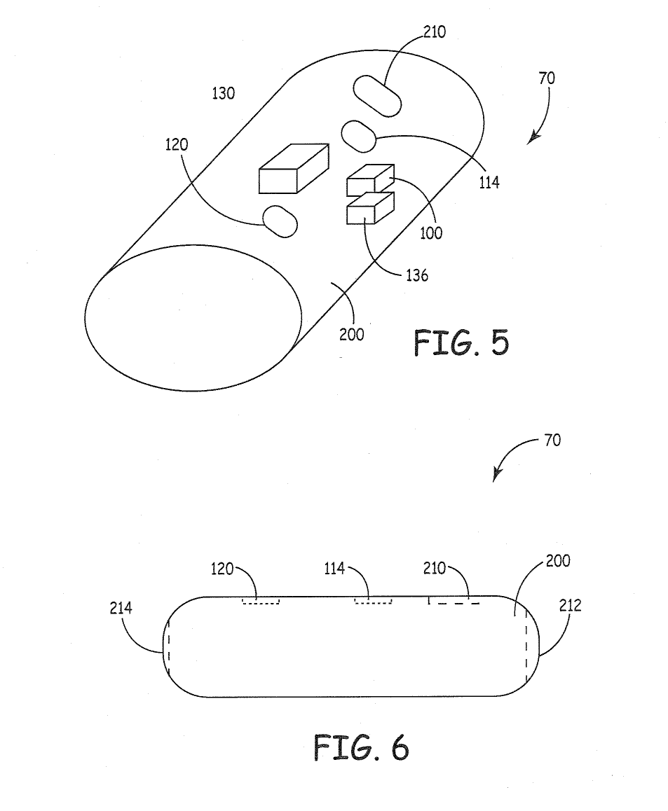

FIGS. 5 and 6 illustrate a perspective and side elevational view of the sensor 70, respectively. The sensor 70 includes a housing 200 and antenna 210 that is coupled with the transmission module 136. While the perfusion sensor 70 has been described as emitting and sensing light, other parameters may also be sensed. For example, electrodes 212, 214 may be disposed on the housing 200 to sense electrical activity (e.g., neurological or cardiac activity). These electrode locations may also be used for alternative emitter/collector locations and as indicated the housing may have many emitter/collector pairs disposed about the entirety of the housing. As illustrated in FIG. 6, the ends of the housing 200 are curved to facilitate implantation, particularly through injection. While the present invention is not limited in terms of specific dimensions, the perfusion sensor 70 has, in one embodiment, a housing 200 with dimensions that permit subcutaneous delivery into tissue through a hypodermic needle. In other embodiments, the housing 70 is delivered via a catheter to a specified location, including transvenous delivery. Finally, the sensor 70 may be implanted directed or via injection during the course of another medical procedure. For example, during organ transplant, the tissue surround the organ is directly exposed to the surgeon and the sensor may be implanted at that time.

Due to the shape and the small size of the housing 200, tissue growth or encapsulation around the housing 200 will minimally interfere with the sensor 70's performance. That is, because the device has curved surfaces and is small compared with the track of the delivery mechanism (e.g., needled), the sensor 70 will be “ignored” by the surrounding tissue as it tends not to irritate that tissue. Thus, whereas some sensors suffer from tissue growth around their implant which would hinder certain optical measurements, the present sensor 70 avoids such issues based upon size and configuration.

Another aspect of the housing 200 is that the sensor 70 may simply be left in place after its use has expired. That is, there is no particular need to extract the device. Furthermore, if monitoring on a longer term basis is required, one embodiment provides for a battery that is externally rechargeable. Alternatively, another device is simply injected proximate the expired device. As such sensors will be relatively inexpensive and may remain implanted, this provides for an effective protocol.

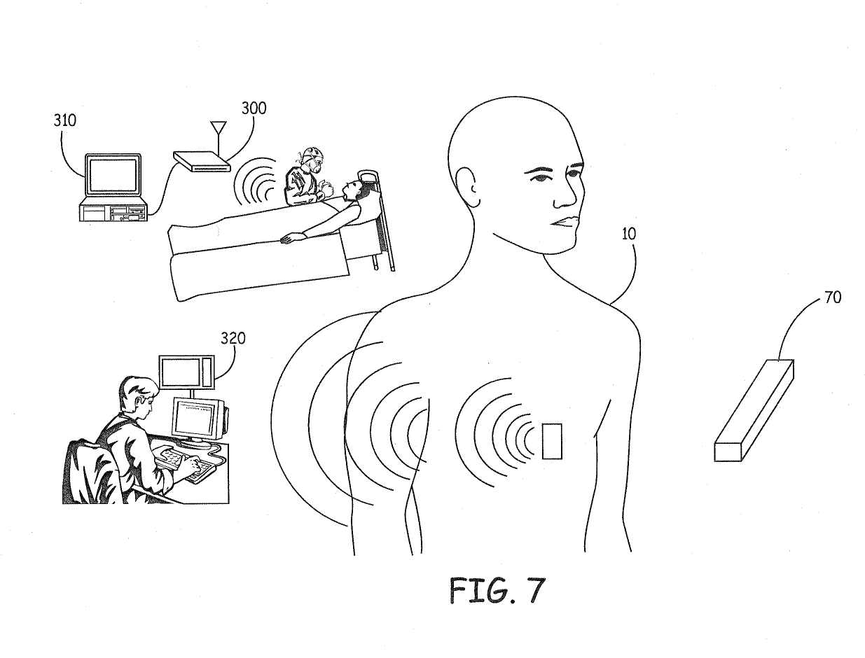

FIG. 7 is a schematic diagram illustrating that patient 10 has a perfusion sensor 70 implanted that transmits data to an external medical device (EMD) 300. The EMD 300 provides that data to an appropriate network 310 where caregivers 320 can access and evaluate the information. It should be appreciated that the data transmitted from the sensor 70 may be raw data that is processed partially or entirely post-transmission. The patient 10 may be under medical supervision; thus, the EMD 300 is located within a medical facility. Alternatively, the patient 10 is ambulatory and the EMD 300 is carried with the patient 10 for continuous data collection or positioned in the patient's home for frequent data collection sessions. Of course, the patient 10 may simply visit a caregiver on a periodic basis to have data from the sensor 70 collected.

The perfusion sensor 70 may be utilized in a variety of ways. In one scenario, a patient may indicate that he has symptom such as pain in an extremity when exposed to cold. The caregiver may prescribe aspirin or other pain relievers to address the pain. Now the caregiver may also inject the wireless perfusion sensor 70. After one or more episodes, the caregiver may evaluate the data and determine whether a transient PVD is present and if so its severity. Furthermore, this disease may progress in severity over time and this is likewise tracked by the sensor 70. Thus, the patient 10 may take the pain medication and tolerate the symptoms and the caregiver can evaluate what is causing the symptoms and if more extensive treatment is required.

In another scenario high risk patients are implanted with one or more wireless perfusion sensors 70 or arrays of such sensors. High risk patients may include, but are not limited to those patients who have previously had an occlusion, including CAD; stroke patient, diabetic patients, and those who have become bedridden or otherwise immobilized. Here, perfusion is monitored and if changes are noted and occlusion or the formation of an occlusion may be detected early, potentially avoiding serious complication.

In another scenario, patients who are being treated for a PVD may be monitored to evaluate the effectiveness of the current treatment. Again, if the treatment is unsuccessful or symptoms redevelop, the sensors 70 will provide an early indication. Likewise, the use of the wireless perfusion sensor may identify the presence of other occlusions.

1. A method for determining an efficacy of an implantable vascular device in a patient having tissue, comprising the steps of:
implanting individual ones of a plurality of perfusion sensors at individual ones of a plurality of sensor locations positioned with respect to said implantable vascular device;
monitoring a perfusion of said tissue proximate said plurality of sensor locations using said plurality of sensors; and
determining an efficacy of said implantable vascular device based, at least in part, on said perfusion of said tissue.

2. The method of claim 1 wherein said implantable vascular device is a vascular stent.

3. The method of claim 2 wherein said vascular stent has a structure and further comprising the step of incorporating one of said plurality of sensors into said structure of said stent.

4. The method of claim 1 further comprising the step of reporting said efficacy of said implantable vascular device to a user.

5. The method of claim 1 wherein at least one of said plurality of locations is distal of said implantable vascular device.

6. The method of claim 5 where at least another one of said plurality of locations is proximate said implantable vascular device.

7. The method of claim 5 wherein said at least one of said plurality of locations is in an extremity of said patient.

8. A method for identifying a location of an occlusion in a vascular system of a patient having a heart, comprising the steps of:
implanting a first sensor and a second sensor proximate said vascular system of said patient, said first sensor being proximal with respect to said heart of said patient relative to said second sensor;
monitoring perfusion of tissue near a location of said first sensor;
monitoring perfusion of tissue near a location of said second sensor; and
determining a location of said collusion based, at least in part, on said perfusion of tissue near said location of said first sensor and said perfusion of tissue near said location of said second sensor.

9. The method of claim 8 wherein said occlusion is determined as being between said first sensor and said second sensor only if said perfusion of said tissue near said second sensor is less than said perfusion of said tissue near said first sensor.

10. The method of claim 8 wherein said first sensor and said second sensor are implanted in an extremity of said patient.

11. The method of claim 8 further comprising the step of reporting said location of said occlusion to a user.

12. The method of claim 8 wherein said first sensor and said second sensor are implanted proximate an artery and wherein if said perfusion of said tissue near said second sensor is approximately equal to said perfusion of said tissue near said first sensor, and said perfusion of said tissue near one of said first sensor and said second sensor is greater than a predetermined threshold, said occlusion being determined to be at a location in said vascular system distal of said heart relative to said first sensor and said second sensor.

13. The method of claim 12 wherein said first sensor and said second sensor are implanted proximate an artery and wherein if said perfusion of said tissue near said second sensor is approximately equal to said perfusion of said tissue near said first sensor, and said perfusion of said tissue near one of said first sensor and said second sensor is less than a predetermined threshold, said occlusion being determined to be at a location in said vascular system proximal to said heart relative to said first sensor and said second sensor.

14. A method for reporting a degree of blockage created by an occlusion in a vascular system of a patient, comprising the steps of:
implanting a plurality of perfusion sensors a plurality of locations in said vascular system;
monitoring a perfusion of tissue of said plurality of locations of said vascular system using said plurality of perfusion sensors; and
determining said degree of blockage created said occlusion based, at least in part, on said perfusion of said tissue.

15. The method of claim 14 further comprising the step of reporting said degree of blockage to a user.

16. A method of determining blood flow in an extremity of a patient having a heart, said extremity having vasculature, comprising the steps of:
implanting a first perfusion sensor and a second perfusion sensor at a first location and a second location, respectively, in said extremity;
monitoring a perfusion of tissue near said first location with said first sensor;
monitoring a perfusion of tissue near said second location with said second sensor; and
determining said blood flow of said extremity based, at least in part, on said perfusion of tissue near said first location and said perfusion of tissue near said second location.

17. The method as in claim 16 wherein said first location and said second location are approximately equidistant vascularly relative to the heart.

18. The method as in claim 16 further comprising the step of reporting said blood flow to a user.

19. The method as in claim 16 wherein said blood flow is indicative of peripheral vascular disease in said extremity.

20. A method for determining a health of an organ of a patient, comprising the steps of:
implanting individual ones of a plurality of perfusion sensors at individual ones of a plurality of locations proximate said organ;
monitoring a perfusion of tissue of at least one of said plurality of locations proximate said organ using at least one of said plurality of perfusion sensors; and
determining said health of said organ based, at least in part, on said perfusion of said tissue.

21. The method of claim 20 wherein said organ is a transplanted organ and said patient has an immune system, and wherein said health of said organ is indicative of rejection by said immune system of said transplanted organ.

22. A method for identifying a location of an occlusion in a vascular system of a patient, comprising the steps of:
implanting a plurality of perfusion sensors at a plurality of locations in said vascular system;
monitoring a perfusion of tissue of said patient at each of said plurality of locations in said vascular system using said plurality of at sensors; and
determining a location of said occlusion based, at least in part, on said perfusion of tissue measured from said plurality of locations.
